(19) United States
(12) Patent Application Publication
(10) Pub. No.: US 2021/0178506 A1
(43) Pub. Date: Jun. 17, 2021

(54) APPARATUS FOR STERILIZED WELDING

(71) Applicant: SAINT-GOBAIN PERFORMANCE PLASTICS CORPORATION, Solon, OH (US)

(72) Inventors: Jianfeng ZHANG, Shrewsbury, MA (US); Rachel Z. Pytel, Newton, MA (US); Anthony P. Pagliaro, Lansdale, PA (US); Clemens E. Zoellner, Bay City, MI (US); Jian L. Ding, Stow, MA (US); Thomas R. Nixon, Au Gres, MI (US)

(21) Appl. No.: 16/952,414

(22) Filed: Nov. 19, 2020

Related U.S. Application Data
(60) Provisional application No. 62/947,203, filed on Dec. 12, 2019.
(60) Provisional application No. 63/041,407, filed on Jun. 19, 2020.

(51) Int. Cl. B23K 10/02; A61L 2/14; B23K 10/00

(52) U.S. Cl. CPC B23K 10/02; B23K 2103/42; B23K 10/006; A61L 2/14

(57) ABSTRACT

Systems and methods are disclosed that include generating a plasma treatment and applying the plasma treatment to end contact surfaces of a first profile and a second profile in a sterile environment, manipulating at least one of the first profile and the second profile to force contact between the end contact surface of the first profile and the end contact surface of the second profile to permanently connect, join, or weld the end contact surfaces of the first profile and the second profile together, thereby forming a sterile connection between the first profile and a second profile.

### CROSS-REFERENCE TO RELATED APPLICATION(S)

This application claims priority under 35 U.S.C. § 119(e) to U.S. Provisional Application No. 62/947,203, entitled “APPARATUS FOR STERILIZED WELDING,” by Jianfeng ZHANG et al., filed Dec. 12, 2019, and to U.S. Provisional Application No. 63/041,407, entitled “APPARATUS FOR STERILIZED WELDING,” by Jianfeng ZHANG et al., filed Jun. 19, 2020, all of which are assigned to the current assignee hereof and incorporated by reference in their entireties.

### BACKGROUND OF THE INVENTION

Sterile connections are used to connect various profiles for the delivery and removal of fluids. Such sterile connections may be used in a variety of industries, including the medical industry and the pharmaceutical industry. Thermoplastic and thermoset elastomers are often used in such applications since these elastomers are non-toxic, flexible, thermally stable, have low chemical reactivity, and can be produced in a variety of sizes. In many instances, it is desirable to connect two different profiles to create a sterile fluid connection. Unfortunately, traditional welding apparatuses that use high temperature methods cannot effectively join thermoset elastomers, such as a silicone elastomer that cannot be melted, to another thermoset elastomer or other polymeric materials. Further, joining such materials also presents challenges in maintaining sterility at the connection.

### SUMMARY

The present disclosure relates generally to a plasma welding apparatus and methods for applying a plasma treatment to end contact surfaces of a first profile and a second profile, preferably in a sterile environment, manipulating at least one of the first profile and the second profile to force contact between the end contact surface of the first profile and the end contact surface of the second profile to permanently connect, join, or weld the end contact surfaces of the first profile and the second profile together, thereby forming a sterile connection between the first profile and a second profile.

### BRIEF DESCRIPTION OF THE DRAWINGS

So that the manner in which the features and advantages of the embodiments are attained and can be understood in more detail, a more particular description may be had by reference to the embodiments thereof that are illustrated in the appended drawings. However, the drawings illustrate only some embodiments and therefore are not to be considered limiting in scope as there may be other equally effective embodiments.

FIG. 1 is a schematic of a plasma welding apparatus according to an embodiment of the disclosure.

FIG. 2 is a schematic diagram of a mechanical motion module according to an embodiment of the disclosure.

FIG. 3 is a schematic diagram of a mechanical motion module according to an embodiment of the disclosure.

FIG. 4 shows a first profile and a second profile in an open position for plasma welding.

FIG. 5 shows a first profile and a second profile in a closed position at a distal end of each of the first profile and the second profile for plasma welding.

FIG. 6 shows a first profile and a second profile in a closed position away from a distal end of each of the first profile and the second profile for plasma welding.

FIG. 7 is a flowchart of a method of operating a plasma welding apparatus according to an embodiment of the disclosure.

FIGS. 8A to 8G are schematic diagrams of a mechanical motion module and showing various steps of operating the mechanical motion module in a welding apparatus according to an according to an embodiment of the disclosure.

FIG. 9 is a schematic diagram of a mechanical motion module according to another embodiment of the disclosure.

FIG. 10 is a schematic diagram of a portion of a mechanical motion module according to an embodiment of the disclosure.

The use of the same reference symbols in different drawings indicates similar or identical items.

### DETAILED DESCRIPTION

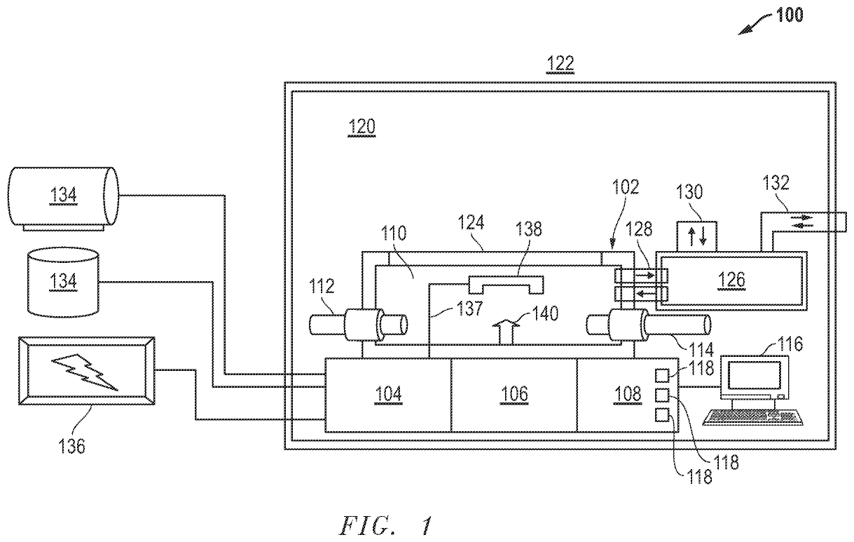

FIG. 1 shows a schematic of a plasma welding apparatus 100 according to an embodiment of the disclosure. Most generally, the plasma welding apparatus 100 may be configured to join two profiles (e.g., tubes, hoses, or other shapes or structures having a lumen through which a fluid may be carried, pumped, or otherwise transported) in a sterile environment. The joining of the two profiles may be accomplished by coincidentally welding a first end of a first profile with a second end of a second profile via a surface activation treatment. The surface activation treatment coincidentally and chemically welds the first end of the first profile to the second end of the second profile when they are placed in direct contact. Any surface activation treatment is envisioned and includes processing input energy to a surface of the first profile, the second profile, or combination thereof. In an embodiment, processing input energy is accomplished by wave irradiation, particular irradiation, or combination thereof. In an embodiment, the wave irradiation includes any wave irradiation envisioned such as radio waves, microwaves, infrared, visible light, ultraviolet, x-rays, gamma radiation, or combination thereof. In a particular embodiment, the wave irradiation includes microwaves, ultraviolet, x-rays, gamma radiation, or combination thereof. In an embodiment, the particle irradiation includes alpha radiation, beta radiation, charged ions, neutron radiation, or combination thereof. The surface activation treatment provides an effective seal between the two profiles.

The plasma welding apparatus 100 may generally comprise an operation chamber 102, a plasma generator 104, a mechanical motion module 106, and a control system 108. The operation chamber 102 may generally comprise an enclosure that defines a substantially sealed, internal environment 110 where a sterile cutting and/or plasma welding operation of a first profile 112 and a second profile 114 may occur. The plasma generator 104 may generally be configured to generate ionized particles through a plasma treatment (e.g., corona treatment, C-treatment, flame treatment, ion treatment, plasma treatment, or combinations thereof) within the operation chamber 102 that may selectively be applied to the first profile 112 and the second profile 114 to activate material on end contact surfaces of the first profile 112 and the second profile 114. The mechanical motion module 106 may generally be configured to axially or otherwise align the first profile 112 and the second profile 114 and/or force contact between the end contact surfaces of the first profile 112 and the second profile 114 to connect, join, or weld the end contact surfaces of the first profile 112 and the second profile 114 together. The control system 108 may generally comprise a user interface 116 and a plurality of sensors 118, indicators, gauges, or a combination thereof to facilitate control, monitoring, and operation of the plasma welding process.

It will be appreciated that traditional welding apparatuses that use high temperature methods cannot effectively join thermoset elastomers, such as a silicone elastomer that cannot be melted, to another thermoset elastomer or other polymeric materials. However, the plasma welding apparatus 100 is configured to connect, join, or weld a wide range of materials. In some embodiments, at least one of the first profile and the second profile may be formed from a polymeric material. However, in some embodiments, both the first profile and the second profile may be formed from a polymeric material. Accordingly, in some embodiments, the first profile and the second profile may be formed from the same polymeric material. In other embodiments, the first profile and the second profile may be formed from different polymeric materials. In some embodiments, the first profile and the second profile may be formed from a thermoplastic elastomer, a thermoset elastomer, or combination thereof. Further, in some embodiments, the thermoplastic elastomer of the first profile and/or the second profile may comprise a polystyrene, a polyester, a silicone copolymer, a silicone thermoplastic vulcanizate, a copolyester, a polyamide, a fluoropolymer, a polyolefin, a polyether-ester copolymer, a thermoplastic urethane, a polyether amide block copolymer, a polyamide copolymer, a styrene block copolymer, a polycarbonate, a thermoplastic vulcanizate, an ionomer, a polyoxymethylene (POM), an acrylonitrile butadiene styrene (ABS), an acetal, an acrylic, a polyvinyl chloride (PVC), a blend, or combination thereof. In some embodiments, the thermoset elastomer of the first profile and/or the second profile may comprise a silicone elastomer, a diene elastomer, a butyl rubber, a natural rubber, a polyurethane rubber, an ethylene propylene diene monomer rubber, an isoprene rubber, a nitrile rubber, a styrene butadiene rubber, a blend, or combination thereof.

In some embodiments, the operation chamber 102 may comprise an enclosure that defines a substantially sealed, internal environment 110 that is separated from an external environment, such as a clean room 120 and/or ambient atmosphere 122 outside the clean room 120 and/or a structure, such as a medical facility. In some embodiments, the operation chamber 102 may be configured to at least partially receive the first profile 112 and the second profile 114 to facilitate the sterile cutting and/or plasma welding operation. However, to maintain a sterile internal environment 110 within the operation chamber 102, the operation chamber 102 may be configured to maintain a fluid tight seal when the first profile 112 and the second profile 114 are received within the operation chamber 102 and partially protrudes therefrom. As such, in some embodiments, the operation chamber may function to confine a plasma or plasma treatment within the internal environment 110.

In some embodiments, the operation chamber 102 may comprise at least one transparent surface 124 through which the first profile 112 and the second profile 114 are visible. In other embodiments, the operation chamber 102 may comprise multiple transparent surfaces 124 through which the first profile 112 and the second profile 114 are visible. In yet other embodiments, the operation chamber 102 may be formed from a transparent material (e.g., acrylic, acrylic glass, plexiglass, polycarbonate, or a combination thereof) that allows observation of the first profile 112 and the second profile 114 by an operator from all angles and/or sides of the operation chamber 102. These embodiments may allow an operator to observe the plasma welding process and/or inspect the final connection between the first profile 112 and the second profile 114 for completion, quality, or a combination thereof. Further, in some embodiments, to protect the safety of an operator and/or integrity of the plasma treatment, the transparent surface(s) may at least partially block ultraviolet light from entering or exiting the enclosure.

As stated, the operation chamber 102 may maintain a substantially sterile, internal environment 110 within the operation chamber 102. In some embodiments, this may be facilitated by a two-way ventilation system 126. In some embodiments, the ventilation system 126 may exchange and/or filter air within the internal environment 110 via a first exchange system 128. In some embodiments, the ventilation system 126 may exchange and/or filter air between the clean room 120 and the internal environment 110 via a second exchange system 130. Further, in some embodiments, the ventilation system 126 may exchange and/or filter air between the ambient atmosphere 122 and the internal environment 110 via a third exchange system 132. As such, the ventilation system 126 may comprise a filter, a catalytic converter, a radiative element, or a combination thereof that treats (e.g., filters, sterilizes, reduces ozone, temperature conditions, or combinations thereof) air received from the clean room 120 via the second exchange system 130 and that enters or exits the internal environment 110 of the operation chamber 102 through the first exchange system 128. Additionally, the ventilation system 126 may also comprise a filter, a catalytic converter, a radiative element, or a combination thereof that treats air received from ambient atmosphere 122 via the third exchange system 132 and that enters or exits the internal environment 110 of the operation chamber 102 through the first exchange system 128.

The plasma generator 104 may generally comprise a gas supply 134, a power supply 136, and at least one plasma head 138 and be configured to generate ionized particles through a plasma treatment (e.g., corona treatment, C-treatment, flame treatment, ion treatment, plasma treatment, or combinations thereof) within the operation chamber 102 that may selectively be applied to the first profile 112 and the second profile 114 to activate material on end contact surfaces of the first profile 112 and the second profile 114. The gas supply 134 may be configured to provide a flow of one or more gasses, such as an inert gas, an oxygen containing gas, a nitrogen containing gas, a fluorine containing gas, or a combination thereof. In some embodiments, the gas supply 134 may comprise an atmospheric air supply, a compressor, a compressed gas cylinder, an in-house gas line, an in-house compressed gas line, a fan, a turbo, or any combination thereof to produce the flow of gas. In some embodiments, the inert gas may comprise argon, neon, helium, or any combination thereof. In other embodiments, the oxygen containing gas may comprise atmospheric air, pure oxygen, alcohol, water vapor, or a combination thereof. In yet other embodiments, the nitrogen containing gas may comprise atmospheric air, pure nitrogen, ammonia, or a combination thereof. Still, in other embodiments, the fluorine containing gas may comprises sulfur hexafluoride (SF6), trifluoromethane (CHF3), tetrafluoromethane (CF4), octafluorocyclobutane (C4F8), or a combination thereof.

The power supply 136 may generally be configured to ionize the flow of gas by imparting an electrical charge to the flow of gas to generate the plasma treatment. In some embodiments, the power supply 136 may be configured to provide an alternating current voltage of at least 110 VAC, at least 120 VAC, at least 220 VAC, or at least 240 VAC. However, in other embodiments, the power supply 136 may be configured to provide a direct current voltage of at least 6 VDC, at least 9 VDC, at least 12 VDC, at least 24 VDC, or at least 48 VDC.

The plasma treatment may be delivered to the at least one plasma head 138 disposed within the internal environment 110 of the operation chamber 102 through at least one supply line 137. The plasma head 138 may be disposed within the internal environment 110 of the operation chamber 102 and located within the operation chamber, such that the at least one plasma head 138 applies the plasma treatment to the end contact surfaces of the first profile 112 and the second profile 114. Accordingly, applying the plasma applying the plasma treatment to the end contact surfaces of the first profile 112 and the second profile 114 may comprise exposing or subjecting the end contact surfaces of the first profile 112 and the second profile 114 to the plasma treatment or delivering or directing the plasma treatment to contact or substantially envelope the end contact surfaces of the first profile 112 and the second profile 114. By applying the plasma treatment to the end contact surfaces, material at the end contact surfaces may be activated for welding the first profile 112 to the second profile 114 when they are forced into contact.

In some embodiments, the plasma generator 104 may comprise a single plasma head 138 disposed within the internal environment 110 of the operation chamber 102. However, in some embodiments, the plasma generator 104 may comprise a plurality of plasma heads 138 disposed within the internal environment 110 of the operation chamber 102. In a particular embodiment, at least one of the plurality of plasma heads 138 may be disposed adjacently to the first profile 112 within the operation chamber 102, and at least one of the plurality of plasma heads 138 may be disposed adjacently to the second profile 114 within the internal environment 110 of the operation chamber 102. Furthermore, in another particular embodiment, at least one of the plurality of plasma heads 138 may be configured to sterilize the internal environment 110 within the operation chamber 102.

The mechanical motion module 106 may generally be configured to axially or otherwise align the first profile 112 and the second profile 114 and/or force contact between the end contact surfaces of the first profile 112 and the second profile 114 to connect, join, or weld the end contact surfaces of the first profile 112 and the second profile 114 together. In some embodiments, the mechanical motion module 106 may be disposed at least partially within the operation chamber 102 or at least partially form a lower barrier or perimeter of the internal environment 110 of the operation chamber 102. In some embodiments, the mechanical motion module 106 may comprise a cutting system 140. However, in some embodiments, the cutting system 140 may be a standalone component. In some embodiments, the cutting system 140 may be disposed in the internal environment 110 of the operation chamber 102. The cutting system 140 may generally be configured to selectively cut a first tubing component to form the first profile 112 and a second tubing component to form the second profile 114. Accordingly, in some embodiments, the cutting system 140 may comprise at least one cutting device, such as at least one blade, and the at least one blade may be heated, pre-sterilized, sterilized by the plasma treatment, or a combination thereof. In some embodiments, where the at least one blade of the cutting system 140 is heated and the first tubing component 111 and the second tubing component 113 are formed from a thermoplastic elastomer, the at least one blade may be used to cut the first tubing component 111 to form the first profile 112 and cut the second tubing component 113 to form the second profile 114, which melts the ends of the profiles 112, 114, and the melted ends of the profiles 112, 114 may be joined without the use of a plasma treatment to the ends of the profiles 112, 114 but in the plasma-sterilized, internal environment 110 of the operation chamber 104. However, in some embodiments, the cutting device may comprise a laser cutting device or system.

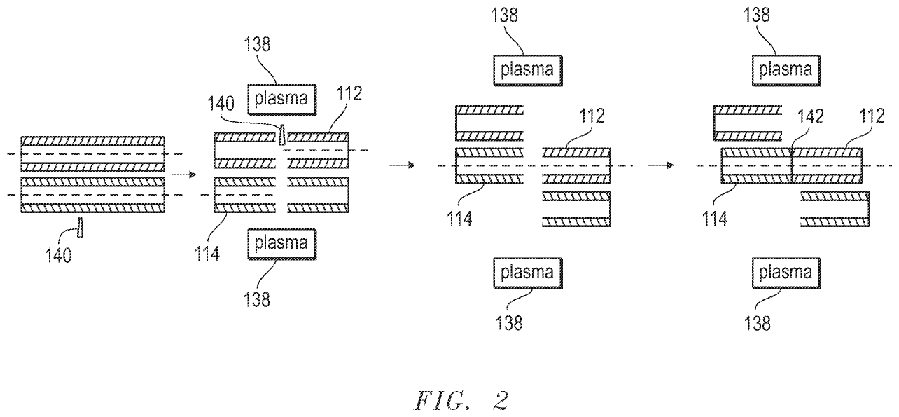

FIG. 2 shows a schematic diagram of a mechanical motion module 106 according to an embodiment of the disclosure. In the embodiment shown, the mechanical motion module 106 comprises a linear motion module. To prepare the first profile 112 and the second profile 114 for plasma welding, the cutting system 140 may be selectively operated to cut two components to form the first profile 112 and the second profile 114. In some embodiments, the plasma treatment may be applied before, during, and/or after the cutting process to activate material on the end contact surfaces of the first profile 112 and the second profile 114. The mechanical motion module 106 may be configured to axially align the end contact surfaces of the first profile 112 and the second profile 114. In some embodiments, the mechanical motion module 106 may linearly displace (substantially perpendicular to axial) at least one of the first profile 112 and the second profile 114 to axially align the end contact surfaces of the first profile 112 and the second profile 114. In other embodiments, the mechanical motion module 106 may linearly displace both the first profile 112 and the second profile 114 to axially align the end contact surfaces of the first profile 112 and the second profile 114. Additionally, once axially aligned, the mechanical motion module 106 may also be configured to force the end contact surfaces, which are activated by the plasma treatment, of the first profile 112 and the second profile 114 into contact to weld the first profile 112 and the second profile 114 at their end contact surfaces, thereby forming a joint or plasma welded connection 142. In some embodiments, the plasma treatment may continue after the first profile 112 and the second profile 114 are joined to ensure a sterile connection, thereby forming a joint or plasma welded connection 142.

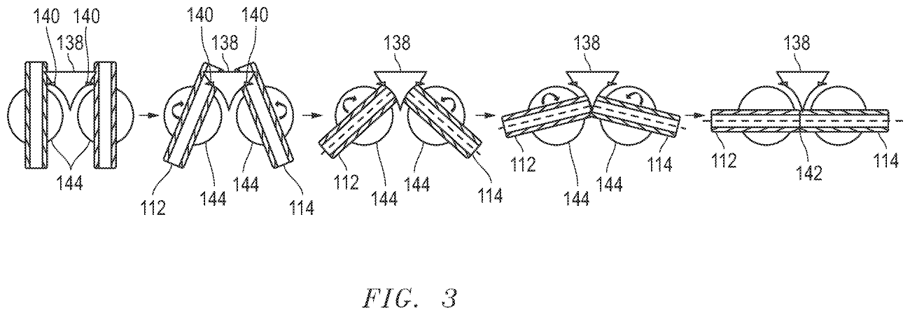

FIG. 3 shows a schematic diagram of a mechanical motion module 106 according to another embodiment of the disclosure. In the embodiment shown, the mechanical motion module 106 comprises a rotary motion module. To prepare the first profile 112 and the second profile 114 for plasma welding, two components captured by rotary motion devices 144 may be selectively rotated to bring the two components into contact with respective cutting devices to form the first profile 112 and the second profile 114. In some embodiments, the plasma treatment may be applied before, during, and/or after the cutting process to activate material on the end contact surfaces of the first profile 112 and the second profile 114. The rotary motion devices 144 of the mechanical motion module 106 may be configured to continually rotate the first profile and the second profile to axially align the end contact surfaces of the first profile 112 and the second profile 114 to axially align the end contact surfaces of the first profile 112 and the second profile 114. In other embodiments, the mechanical motion module 106 may linearly displace both the first profile 112 and the second profile 114 to axially align the end contact surfaces of the first profile 112 and the second profile 114. Additionally, once axially aligned, the rotary motion devices 144 of the mechanical motion module 106 may also be configured to force the end contact surfaces, which are activated by the plasma treatment, of the first profile 112 and the second profile 114 into contact to weld the first profile 112 and the second profile 114 at their end contact surfaces, thereby forming a joint or plasma welded connection 142. In some embodiments, the plasma treatment may continue after the first profile 112 and the second profile 114 are joined to ensure a sterile joint or plasma welded connection 142.

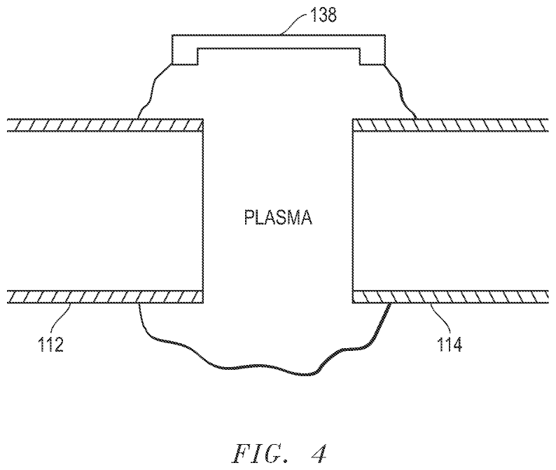

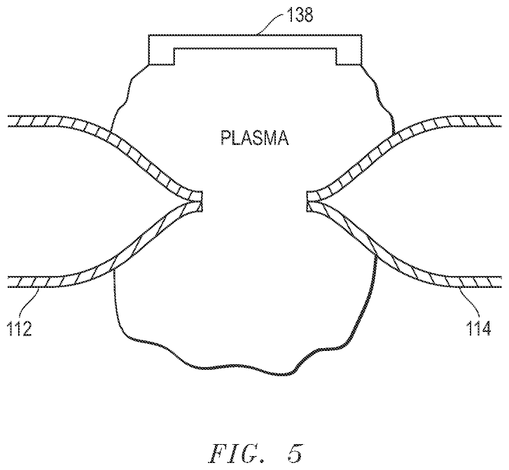

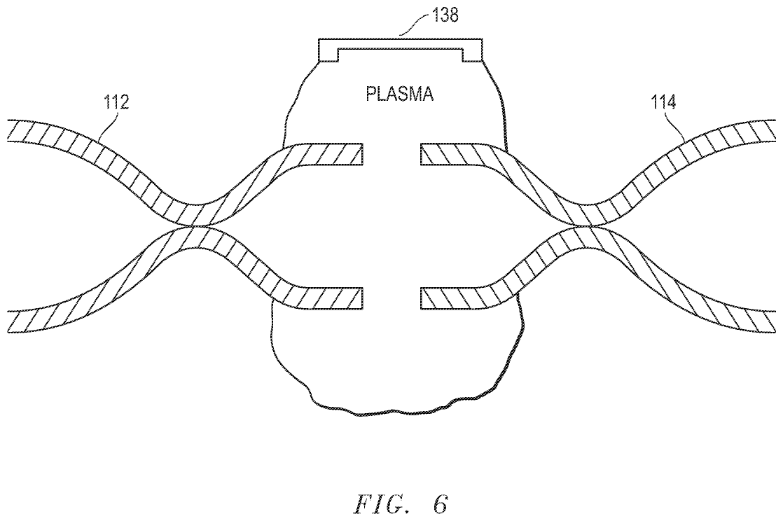

In some embodiments, the mechanical motion module 106 may be configured to manipulate one or more lumens of the first profile 112 and the second profile 114 prior to plasma welding. As such in some embodiments, the first profile 112 and the second profile 114 may remain open during the plasma treatment and/or joining the first profile 112 and the second profile 114. FIG. 4 shows the first profile 112 and the second profile 114 in an open position for plasma welding. In other embodiments, the mechanical motion module 106 may at least partially close one or more of the lumens of the first profile 112 and the second profile 114 prior to plasma welding. In some embodiments, the first profile 112 and the second profile 114 may be closed and/or sealed prior to joining the first profile 112 and the second profile 114. FIG. 5 shows the first profile 112 and the second profile 114 in a closed position at a distal end of each of the first profile 112 and the second profile 114 for plasma welding. FIG. 6 shows the first profile 112 and the second profile 114 in a closed position away from a distal end of each of the first profile 112 and the second profile 114 for plasma welding.

As stated, the plasma treatment may be applied before, during, and/or after the cutting process and/or the joining process to activate material on the end contact surfaces of the first profile 112 and the second profile 114. In some embodiments, the plasma treatment may be applied before cutting the first profile 112 and the second profile to ensure a sterile connection. In some embodiments, the plasma treatment may begin at least 1 second, at least 2 seconds, at least 3 seconds, at least 5 seconds, at least 10 seconds, at least 15 seconds at least 30 seconds, at least 45 seconds at least 60 seconds, at least 90 seconds, or at least 120 seconds before cutting the first profile 112 and the second profile 114. In some embodiments, the plasma treatment may continue after the first profile and the second profile are joined to ensure a sterile connection. In some embodiments, the plasma treatment may continue for at least 1 second, at least 2 seconds, at least 3 seconds, at least 5 seconds, at least 10 seconds, at least 15 seconds at least 30 seconds, at least 45 seconds at least 60 seconds, at least 90 seconds, or at least 120 seconds after joining the first profile 112 and the second profile 114.

Ensuring a sterile connection between the first profile 112 and the second profile 114 may be the result of pretreating the internal environment 110 of the operation chamber 104 with the plasma treatment or beginning the plasma treatment before the cutting operation of the first profile 112 and the second profile 114. Further, ensuring the sterile connection between the first profile 112 and the second profile 114 may also be the result of continuing to apply the plasma treatment after the first profile 112 and the second profile 114 are joined via plasma welding. Accordingly, the plasma treatment provides an interior environment 110 of the operation chamber 102 with a sterile environment, wherein the sterile environment is defined by a reduction in the amount of living microorganisms within the interior environment 110 of the operation chamber by a level of at least 10⁶ after an exposure to the plasma treatment or plasma for at least 10 seconds, at least 15 seconds, at least 20 seconds, at least 25 seconds, at least 30 seconds or at least 60 seconds.

Furthermore, it will be appreciated that the plasma welding process may be performed at various temperatures within the internal environment 110 of the operation chamber 102. For example, in some embodiments, the plasma welding process may be performed at room temperature, such as that within the clean room 120. However, it will be appreciated that the plasma welding process may be performed and configured to provide a sterile connection between the first profile 112 and the second profile 114 at any temperature between about 10 degrees Celsius to 350 degrees Celsius.

The control system 108 may generally comprise a user interface 116 and a plurality of sensors 118, indicators, gauges, or a combination thereof to facilitate control, monitoring, and operation of the plasma generator 104, the mechanical motion module 106, and the plasma welding process. The user interface 116 may generally comprise a display configured to display a temperature, a gas flow rate, a gas pressure, a gas detection level, a material of the first profile and the second profile, a plasma treatment progress level, a working cycle, a total number of working cycles, or a combination thereof. In a particular embodiment, the user interface 116 may comprise a material selection input for selecting properties a material of each of the first profile 112 and the second profile 114. Furthermore, in some embodiments, the user interface 116 may comprise a warning system configured to alert an operator when an out of conformance condition exists. The user interface 116 may also be configured to automatically stop the plasma treatment when an out of conformance condition exists. Example out of conformance conditions include, but are not limited to a low temperature, a high temperature, a low pressure, a high pressure, a low gas flow rate, a high gas flow rate, a detection of a plasma byproduct, an invalid selection of a material, a leak in the operation chamber, or a combination thereof. When such conditions occur, the control system 108 may prevent opening and/or removal of the operation chamber 102. Further, the control system 108 may also prevent opening and/or removal of the operation chamber 102 during application of the plasma treatment.

The plurality of sensors 118, indicators, gauges, or combinations thereof may generally be configured to convey data (e.g., operational parameters) regarding the plasma welding operation to an operator and allow an operator to oversee the plasma welding process. In some embodiments, the plurality of sensors 118 may comprise a temperature sensor, a gas flow rate sensor, a gas pressure sensor, a gas detection sensor, a plasma byproduct sensor, a tension sensor, or a combination thereof. Further, in some embodiments, the control system 108 may be configured to log or store data transmitted from the sensors 118 related to operation of the plasma welding apparatus 100. This data may be used in troubleshooting and/or adjusting operational parameters of the components 102, 104, 106, 108 of the plasma welding apparatus 100 or the plasma welding process.

Furthermore, in some embodiments, the control system 108 may comprise at least one vision system (e.g., camera, inspection, video) configured to verify axial alignment of the end contact surfaces of the first profile 112 and the second profile 114, confirm successful joining of the first profile 112 to the second profile 114, or a combination thereof. In some embodiments, confirming successful joining of the first profile 112 to the second profile 114 may be accomplished by inspection of the welded profiles at an angle (e.g., 30 degrees, 45 degrees), where unsuccessfully joined areas appear darker than successfully joined areas. Further, in some embodiments, the control system 108 may also comprise a marking system configured to mark the first profile 112, the second profile 114, a joint or welded connection 142 formed between the first profile 112 and the second profile 114, or a combination thereof via laser marking, ink marking, or any combination thereof that allows identification, verification, troubleshooting or any combination thereof of one or more characteristics of the joint or welded connection 142 formed between the first profile 112 and the second profile 114.

The plasma welding apparatus 100 may be configured to form a sterile, plasma welded connection 142 between the first profile 112 and the second profile 114. The resulting plasma welded connection 142 may retain specific performance characteristics commensurate with an unmodified control bulk material of the first profile 112 or the second profile 114. Accordingly, in some embodiments, after the joint or plasma welded connection 142 between the first profile 112 and the second profile 114 is formed, the control system 108 may be configured to conduct a burst test, a tension test, or a combination thereof between the first profile 112 and the second profile 114 to ensure a successful plasma welded connection 142 between the first profile 112 and the second profile 114. In some embodiments, a joint or welded connection 142 formed between the first profile 112 and the second profile 114 may comprise a tensile strength of at least 10%, at least 15%, at least 25%, at least 30%, at least 35%, at least 40%, at least 45%, or at least 50% of the tensile strength of an unmodified control bulk material of the first profile 112 or the second profile 114. Furthermore, in some embodiments, a joint or welded connection 142 formed between the first profile 112 and the second profile 114 may comprise a burst pressure of at least 10%, at least 25%, at least 50%, at least 60%, at least 65%, at least 70%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, or at least 90% of the of the burst pressure of an unmodified control bulk material of the first profile or the second profile.

Still further, in some embodiments, a reinforcement may be at least partially applied about the plasma welded connection 142 formed between the first profile 112 and the second profile 114. The reinforcement may comprise an adhesive tape, a polymer tape, an overmolded polymer, a plasma-welded polymer or polymer tape, or a combination thereof.

The plasma welding apparatus 100 may generally be configured such that the operation chamber 102 and the mechanical motion module 106 are configured for mobile use and remotely coupled to the plasma generator 104 and/or the control system 108. Additionally, in some embodiments, the plasma welding apparatus 100 may be a unitary apparatus mounted to a cart or other apparatus for mobile utility, in which the entire plasma welding apparatus 100 may be moved or relocated.

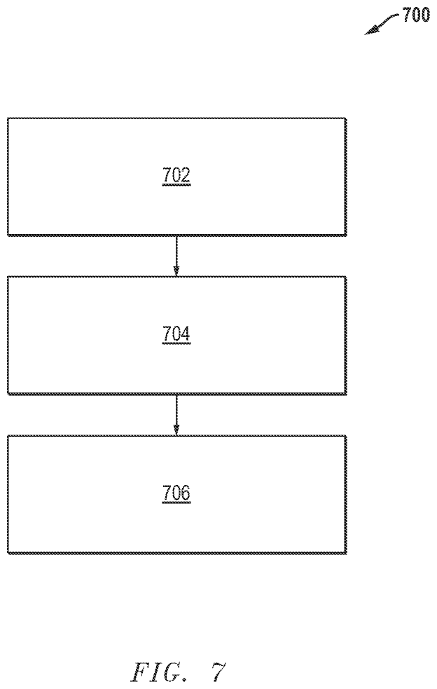

Referring to FIG. 7, a flowchart of a method 700 of operating a plasma welding apparatus 100 is shown according to an embodiment of the disclosure. The method 700 may begin at block 702 by generating a plasma treatment with a plasma generator 104. The method 700 may continue at block 704 by applying the plasma to an end contact surface of a first profile 112 and an end contact surface of a second profile 114 within an operation chamber 102. The method 700 may continue at block 706 by manipulating at least one of the first profile 112 and the second profile 114 to force contact between the end contact surface of the first profile 112 and the end contact surface of the second profile 114 to join the first profile 112 and the second profile 114.

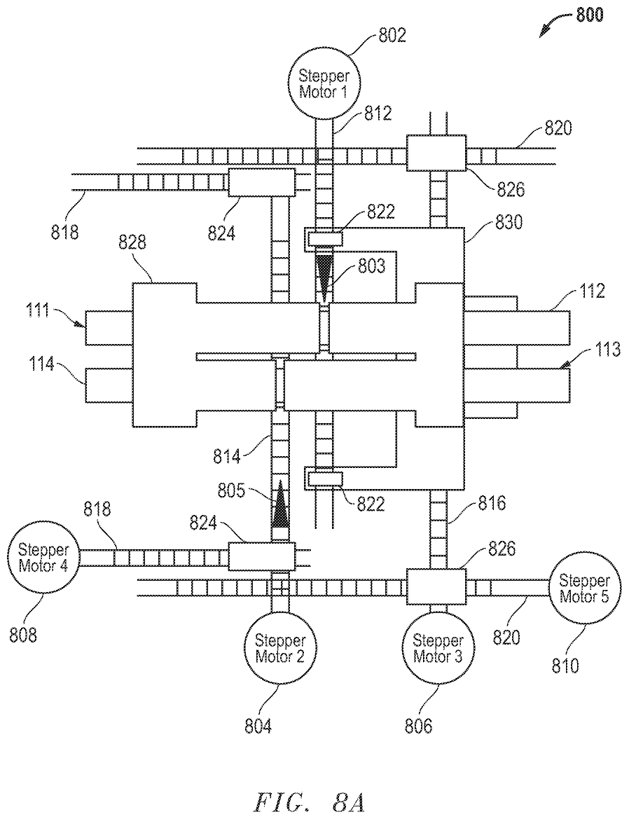

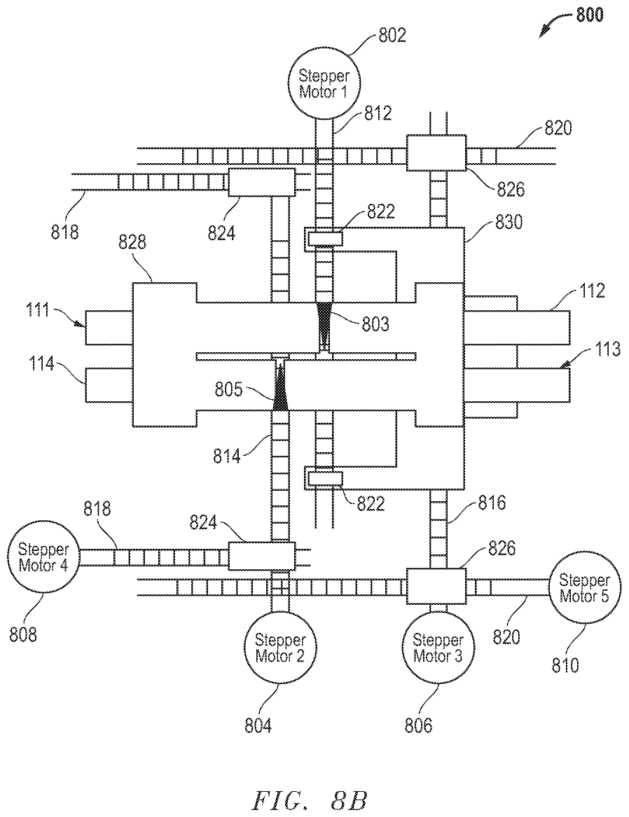

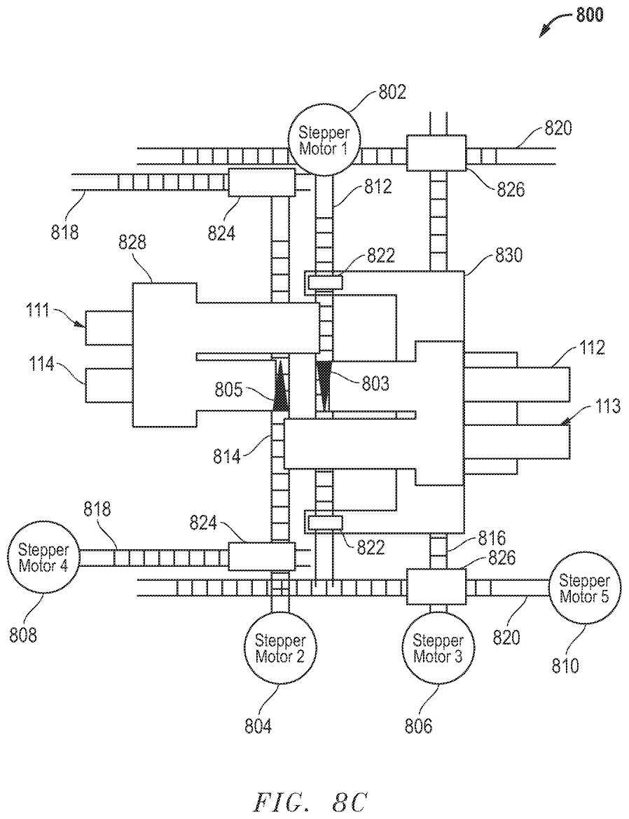

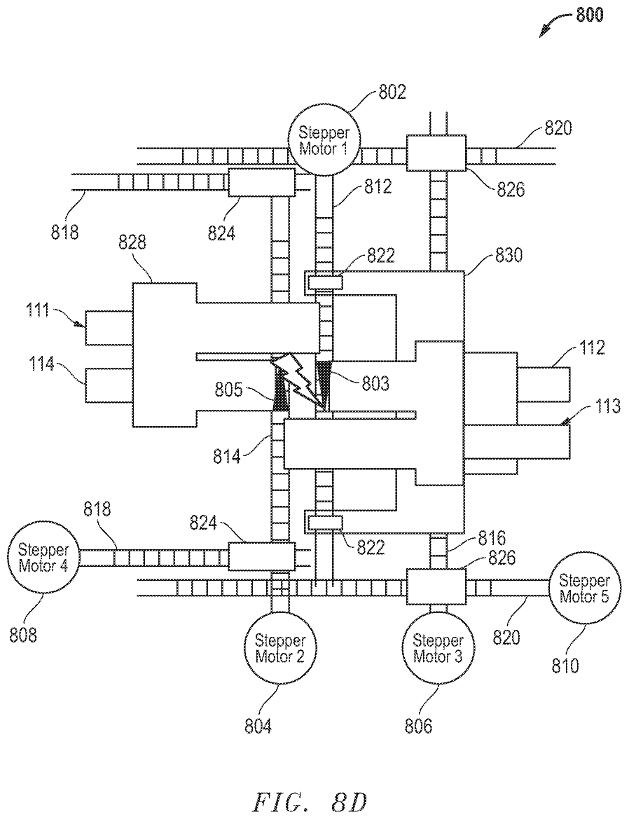

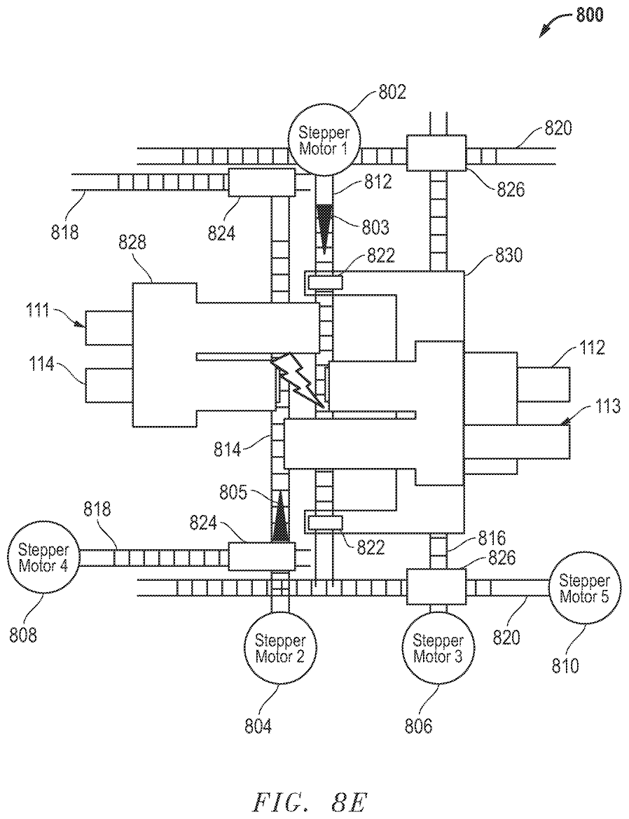

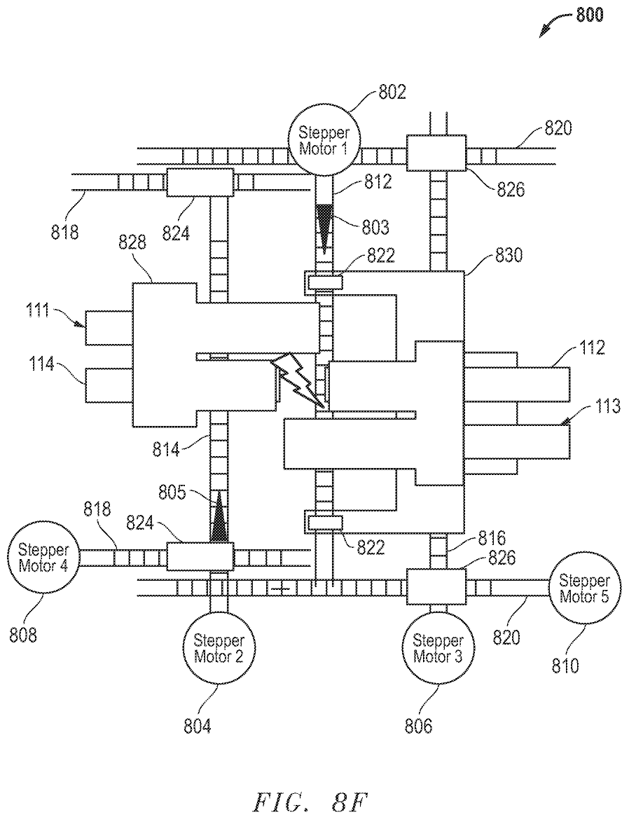

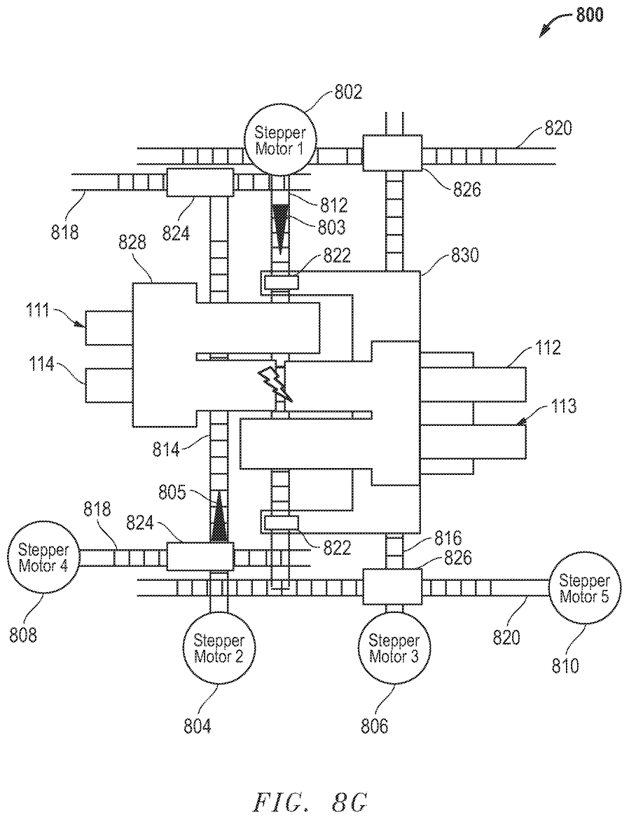

FIGS. 8A to 8G are schematic diagrams of a mechanical motion module 800 and showing various steps of operating the mechanical motion module 800 in a plasma welding apparatus 100 according to an according to an embodiment of the disclosure. The mechanical motion module 800 may generally be substantially similar to the mechanical motion module 106 and configured for operation in a plasma welding apparatus 100 in accordance with embodiments disclosed herein. As such, the mechanical motion module 800 may generally be configured to selectively cut a first tubing component 111 to form a first profile 112 and a second tubing component 113 to form a second profile 114, axially align the first profile 112 and the second profile 114 and force contact between the end contact surfaces of the first profile 112 and the second profile 114 to connect, join, or weld the end contact surfaces of the first profile 112 and the second profile 114 together when subjected to a plasma treatment. Further, it will be appreciated that in some embodiments, the mechanical motion module 800 may be disposed at least partially within the operation chamber 102 of the plasma welding apparatus 100 or at least partially form a lower barrier or perimeter of the internal environment 110 of the operation chamber 102 of the plasma welding apparatus 100.

The mechanical motion module 800 may comprise a cutting system that may be substantially similar to the cutting system 140. In some embodiments, the cutting system may be configured to selectively cut a first tubing component 111 to form the first profile 112 and a second tubing component 113 to form the second profile 114. The cutting system may generally comprise at least one cutting device 803, 805, and the cutting devices 803, 805 may comprise a blade or a laser cutting device or system. In some embodiments, the cutting system may comprise a first cutting device 803 and a second cutting device 805. More specifically, the first cutting device 803 may be configured to cut the first tubing component 111 to form the first profile 112, and the second cutting device may be configured to cut the second tubing component 113 to form the second profile 114. Cutting the first profile 112 and the second profile 114 may generally expose the end contact surfaces of the first profile 112 and the second profile 114 in accordance with embodiments disclosed herein. Further, in some embodiments, the first cutting device 803 and the second cutting device 805 may be heated, pre-sterilized, or a combination thereof, in accordance with embodiments disclosed herein. In some embodiments, where the at least one blade of the cutting system 140 is heated and the first tubing component 111 and the second tubing component 113 are formed from a thermoplastic elastomer, the at least one blade may be used to cut the first tubing component 111 to form the first profile 112 and cut the second tubing component 113 to form the second profile 114, which melts the ends of the profiles 112, 114, and the melted ends of the profiles 112, 114 may be joined without the use of a plasma treatment to the ends of the profiles 112, 114 but in the plasma-sterilized, internal environment 110 of the operation chamber 104.

The mechanical motion module 800 may comprise a plurality of actuation devices 802, 804, 806, 808, 810 configured to move various components of the mechanical motion module 800 along a plurality of rails or tracks 812, 814, 816, 818, 820 to perform the plasma welding process. In some embodiments, the actuation devices 802, 804, 806, 808, 810 may comprise an electrical stepper motor, an electro-mechanical actuator, a pneumatic actuator or cylinder, or any other suitable actuation module that may move components of the mechanical motion module 800 along the plurality of tracks 812, 814, 816, 818, 820. In some embodiments, each actuation device 802, 804, 806, 808, 810 may comprise a sensor, such as sensor 118, that is coupled to the control system 108 of the plasma welding apparatus 100. In some embodiments, each sensor may monitor and relay information associated with operation of the actuation devices 802, 804, 806, 808, 810 to the control system 118. In some embodiments, the information relayed by the sensors to the control system 108 may include directional information, positional information, and/or other operational information. In some embodiments, the plurality of tracks 812, 814, 816, 818, 820 may comprise a plurality of guides 822, 824, 826. The guides may generally be configured to control relative motion and/or maintain proper alignment of the components along the plurality of tracks 812, 814, 816, 818, 820.

The mechanical motion module 800 may also comprise a stationary tubing holder 828 and a movable tubing holder 830. The stationary tubing holder 828 may be configured to remain stationary and secure the first tubing component 111 and the second tubing component 113 in the mechanical motion module 800. In some embodiments, the stationary tubing holder 828 and the movable tubing holder 830 may extend at least partially circumferentially around each of the first tubing component 111 and the second tubing component 113 by at least 25%, at least 50%, or at least 75%. However, in some embodiments, the stationary tubing holder 828 and the movable tubing holder 830 may extend fully circumferentially about each tubing component 111, 113. In some embodiments, the stationary tubing holder 828 may comprise adjustable tubing clamps that allow for various sized tubing components 111,113 to be inserted within the stationary tubing holder 828. The movable tubing holder 830 may also be configured to secure the first tubing component 111 and the second tubing component 113 in the mechanical motion module 800. Once the first tubing component 111 and the second tubing component 113 are cut to form the first profile 112 and the second profile 114, respectively, the movable tubing holder 114 may also be configured to move both axially and transversally to axially align the end contact surfaces of the first profile 112 and the second profile 114 in order to perform the welding operation.

FIGS. 8A to 8G further depict the various steps of operating the mechanical motion module 800 in a plasma welding apparatus 100 according to an according to an embodiment of the disclosure. As shown in FIG. 8A, a first profile 112 and a second profile 114 have been inserted into the stationary tubing holder 828 and the movable tubing holder 830 and prepared to undergo a welding operation. In some embodiments, the stationary tubing holder 828 and the movable tubing holder 830 may be spaced apart a predetermined distance prior to cutting the first component 111 to form the first profile 112 and the second component 113 to form the second profile 114. In some embodiments, the predetermined distance between the stationary tubing holder 828 and the movable tubing holder 830 may be at least 1.0 mm, at least 2.0 mm, at least 3.0 mm, at least 4.0 mm, or at least 5.0 mm. In some embodiments, the predetermined distance between the stationary tubing holder 828 and the movable tubing holder 830 may be not greater than 15.0 mm, not greater than 14.0 mm, not greater than 13.0 mm, not greater than 12.0 mm, not greater than 11.0 mm, or not greater than 10.0 mm. Further, it will be appreciated that the predetermined distance between the stationary tubing holder 828 and the movable tubing holder 830 may be between any of these minimum and maximum values, such as at least 1.0 mm to not greater than 15.0 mm, or even at least 5.0 mm to not greater than 10.0 mm.

As shown in FIG. 8B, the first actuation device 802 may generally be operated to transversally move the first cutting device 803 along the first track 812 to cut the first profile 112. Guides 822 coupled to the movable tubing holder 830 may maintain proper alignment of the first cutting device 803 and the first profile 112. The second actuation device 804 may generally be operated to transversally move the second cutting device 805 along the second track 814 to cut the second profile 114. Guides 824 may maintain proper alignment of the second cutting device 805 and the second profile 114. In some embodiments, the first cutting device 803 and the second cutting device 805 may be aligned in the transversal direction. However, in other embodiments, the first cutting device 803 and the second cutting device 805 may be misaligned in the transversal direction. In some embodiments, the first cutting device 803 and the second cutting device 805 may be configured to cut the first profile 112 and the second profile 114 at an angle less than 90 degrees with respect to a length of the first profile 112 and the second profile 114. However, in other embodiments, the first cutting device 803 and the second cutting device 805 may be configured to cut the first profile 112 and the second profile 114 at a substantially orthogonally with respect to a length of the first profile 112 and the second profile 114.

As shown in FIG. 8C, the third actuation device may generally be operated to transversally move the movable tubing holder 830 along the third track 816 to axially align the first profile 112 and the second profile 114. During movement of the movable tubing holder 830, the stationary tubing holder 828 remains stationary, so that the movable tubing holder 830 displaces transversally with respect to the stationary tubing holder 828 to axially align the first profile 112 and the second profile 114.

As shown in FIG. 8D, the plasma treatment may be applied to the first profile 112 and the second profile 114. More specifically, in some embodiments, the plasma treatment may be applied to the first profile 112 and the second profile 114 to activate material on the end contact surfaces of the first profile 112 and the second profile 114 in accordance with embodiments disclosed herein. Further, it will be appreciated that in some embodiments, the plasma treatment may be applied before, during, and/or after the cutting process.

As shown in FIG. 8E, once the plasma treatment has been activated, the first actuation device 802 may generally be operated to retract the first cutting device 803 along track 812 to expose the end contact surface and/or the lumen of the first profile 112 to the plasma treatment, and the second actuation device 804 may generally be operated to retract the second cutting device 805 along track 814 to expose the end contact surface and/or the lumen of the second profile 114 to the plasma treatment. It will be appreciated that the plasma treatment may remain activated during this step in the welding process.

As shown in FIG. 8F, after retracting the second cutting device 805, the fourth actuation device 808 may generally be operated to axially move the second cutting device 805 along the fourth track 818. In some embodiments, the axial movement of the second cutting device 805 may provide clearance for axial movement of the movable tubing holder 830. It will be appreciated that the plasma treatment may remain activated during this step in the welding process.

As shown in FIG. 8G, after axially moving the second cutting device 805, the fifth actuation device 810 may generally be operated to axially move the movable tubing holder 830 along the fifth track 820 to force the end contact surfaces, which are activated by the plasma treatment, of the first profile 112 and the second profile 114 into contact to weld the first profile 112 and the second profile 114 at their end contact surfaces, thereby forming a joint or plasma welded connection. In some embodiments, the plasma treatment may continue after the first profile 112 and the second profile 114 are joined to ensure a sterile connection, thereby forming a joint or plasma welded connection in accordance with embodiments disclosed herein.

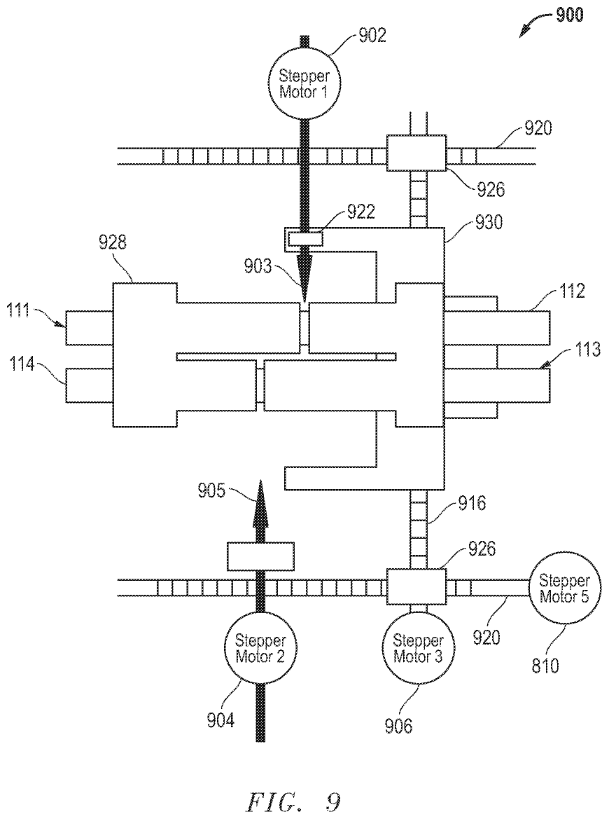

FIG. 9 shows a schematic diagram of a mechanical motion module 900 according to another embodiment of the disclosure. The mechanical motion module 900 may generally be substantially similar to the mechanical motion module 800 and configured for operation in a plasma welding apparatus 100 in accordance with embodiments disclosed herein. Similarly, the mechanical motion module 900 comprise a first cutting device 910, a second cutting device 905, a plurality of actuation devices 902, 904, 906, 910 configured to move various components of the mechanical motion module 800 along a plurality of rails or tracks 916, 918, 920, a stationary tubing holder 928, and a dynamic tubing holder 930. In some embodiments, the mechanical motion module 900 may also comprise a pair of guides 926. However, in the embodiment depicted, the mechanical motion module 900 may not include the fourth actuation device 808, the first track 812, the second track 814, the fourth track 818, and/or the guides 824. Instead, the second cutting device 905 is translated axially by the fifth actuation device 910 to provide clearance for axial movement of the movable tubing holder 930. Accordingly, in some embodiments, this may simplify operation of the welding process by eliminating the step of axially moving the second cutting device 905 (as shown in FIG. 8G) to provide clearance for axial movement of the movable tubing holder 930.

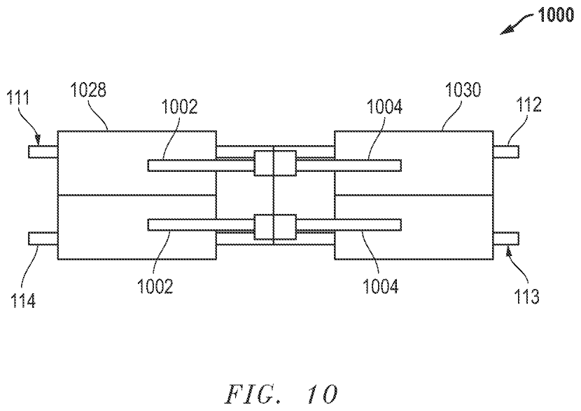

FIG. 10 is a schematic diagram of a portion of a mechanical motion module 1000 according to an embodiment of the disclosure. In some embodiments, the mechanical motion module 1000 may comprise one or more alignment rods 1002, 1004. More specifically, in the embodiment shown, the stationary tubing holder 1028 may comprise one or more alignment rods 1002, and the movable tubing holder 1030 may comprise one or more alignment rods 1004. In some embodiments, each tubing component 111, 113 may be supported by an alignment rod 1002 of the stationary tubing holder 1028 and an alignment rod of the movable tubing holder 1030 prior to cutting the tubing components 111, 113 to form the profiles 112, 114. In some embodiments, the one or more alignment rods 1002 of the stationary tubing holder 1028 and the one or more alignment rods 1004 of the movable tubing holder 1030 may be configured to abut, engage, or otherwise interact to align the stationary tubing holder 1028 and the movable tubing holder 1030. In some embodiments, the one or more alignment rods 1002 of the stationary tubing holder 1028 and the one or more alignment rods 1004 of the movable tubing holder 1030 may be configured to selectively extend and guide the first profile 112 and the second profile 114 into contact with one another such that the end contact surfaces of the first profile 112 and the second profile 114 make contact and the first profile 112 and the second profile 114 are axially aligned. In some embodiments, this may be accomplished during activation or application of the plasma treatment. Further, in some embodiments, the one or more alignment rods 1002 of the stationary tubing holder 1028 and the one or more alignment rods 1004 of the movable tubing holder 1030 may be configured to selectively extend and support the first tubing component 111 and the second component 113 prior to the cutting operation that forms the first profile 112 and the second profile 114.

In some embodiments, the mechanical motion modules 106, 800, 900, 1000 may comprise more than one of the components depicted. For example, in some embodiments, the mechanical motion module 800 may comprise two stationary tubing holder 828 and two movable tubing holders 830, such that four tubing components 111, 113 may be cut and two profiles 112, 114 may undergo a plasma welding process to form two plasma welded tubes.

Further, in some embodiments, a plasma welding apparatus 100 may comprise a plurality of one or more mechanical motion modules 106, 800, 900, 1000. In some embodiments, the plurality of mechanical motion modules 106, 800, 900, 1000 may be standalone systems or may be at least partially integrated. In some embodiments, the plurality of mechanical motion modules 106, 800, 900, 1000 may be configured to cut a plurality of tubing components 111, 113 simultaneously and weld a plurality of profiles, 112, 114.

In some embodiments, the method 700 may comprise one or more of the following: ionizing a flow of gas comprising an inert gas, an oxygen containing gas, a nitrogen containing gas, a fluorine containing gas, or a combination thereof to generate the plasma treatment; sterilizing the internal environment 110 within the operation chamber 102 by applying the plasma treatment within the operation chamber 102; cutting the first profile 112 and the second profile 114 to expose the end contact surfaces of the first profile and the second profile; applying the plasma treatment to the first profile 112 and the second profile 114 prior to cutting the first profile 112 and the second profile 114, wherein the plasma is applied to the first profile and the second profile at least 1 second, at least 2 seconds, at least 3 seconds, at least 5 seconds, at least 10 seconds, at least 15 seconds at least 30 seconds, at least 45 seconds at least 60 seconds, at least 90 seconds, or at least 120 seconds before cutting the first profile 112 and the second profile 114; and applying the plasma to the first profile 112 and the second profile 114 after joining the first profile 112 and the second profile 114, wherein the plasma is applied to the first profile and the second at least 1 second, at least 2 seconds, at least 3 seconds, at least 5 seconds, at least 10 seconds, at least 15 seconds at least 30 seconds, at least 45 seconds at least 60 seconds, at least 90 seconds, or at least 120 seconds after joining the first profile 112 and the second profile 114.

In some embodiments, the method 700 may also comprise one or more of the following: axially aligning the end contact surfaces of the first profile 112 and the second profile 114 after cutting the first profile 112 and the second profile 114; verifying the axial alignment of the end contact surfaces of the first profile 112 and the second profile 114 via a camera system; monitoring a temperature, a gas flow rate, a gas pressure, a gas detection level, a material of the first profile and the second profile, a plasma treatment progress level, a working cycle, a total number of working cycles, or a combination thereof; displaying the temperature, the gas flow rate, the gas pressure, the gas detection level, the material of the first profile 112 and the second profile 114, the plasma treatment progress level, the working cycle, the total number of working cycles, or a combination thereof on a user interface; alerting a user when an out of conformance condition exists; automatically stopping the plasma treatment when an out of conformance condition exists, wherein the out of conformance condition comprises a low temperature, a high temperature, a low pressure, a high pressure, a low gas flow rate, a high gas flow rate, a detection of a plasma byproduct, an invalid selection of a material, a leak in the operation chamber, or a combination thereof; storing data related to operation of the plasma welding apparatus 100; conducting a burst test, a tension test, or a combination thereof between the first profile and the second profile test after the first profile and the second profile are joined; selecting a material of each of the first profile 112 and the second profile 114 via the user interface; and applying a reinforcement at least partially about the coincident weld formed between the first profile and the second profile, wherein the reinforcement comprises an adhesive tape, a polymer tape, an overmolded polymer, a plasma-welded polymer, or a combination thereof.

In still other embodiments, the plasma welding apparatus may include one or more of the following items:

Embodiment 1. A plasma welding apparatus, comprising: an operation chamber; and a plasma generator having at least one plasma head disposed within the operation chamber and in proximity to an end contact surface of a first profile within the operation chamber and an end contact surface of a second profile within the operation chamber, wherein the plasma head is configured to apply a plasma treatment to the end contact surfaces of the first profile and the second profile to join the first profile and the second profile.

Embodiment 2. A plasma welding apparatus for sterile connection of a first profile and a second profile, comprising: an operation chamber comprising a sterile environment; and a plasma generator having at least one plasma head disposed within the operation chamber and in proximity to an end contact surface of a first profile within the operation chamber and an end contact surface of a second profile within the operation chamber, wherein the plasma head is configured to apply a plasma treatment to the end contact surfaces of the first profile and the second profile to join the first profile and the second profile.

Embodiment 3. The plasma welding apparatus of any of Embodiments 1 to 2, wherein at least one of the first profile and the second profile are formed from a polymeric material.

Embodiment 4. The plasma welding apparatus of any of Embodiments 1 to 3, wherein each of the first profile and the second profile are formed from a polymeric material.

Embodiment 5. The plasma welding apparatus of Embodiment 4, wherein the first profile and the second profile are formed from the same polymeric material.

Embodiment 6. The plasma welding apparatus of Embodiment 4, wherein the first profile and the second profile are formed from different polymeric materials.

Embodiment 7. The plasma welding apparatus of any of Embodiments 1 to 6, wherein the first profile and the second profile are formed from a thermoplastic elastomer, a thermoset elastomer, or combination thereof.

Embodiment 8. The plasma welding apparatus of Embodiment 7, wherein the thermoplastic elastomer comprises a polystyrene, a polyester, a silicone copolymer, a silicone thermoplastic vulcanizate, a copolyester, a polyamide, a fluoropolymer, a polyolefin, a polyether-ester copolymer, a thermoplastic urethane, a polyether amide block copolymer, a polyamide copolymer, a styrene block copolymer, a polycarbonate, a thermoplastic vulcanizate, an ionomer, a polyoxymethylene (POM), an acrylonitrile butadiene styrene (ABS), an acetal, an acrylic, a polyvinyl chloride (PVC), a blend, or combination thereof.

Embodiment 9. The plasma welding apparatus of Embodiment 7, wherein the thermoset elastomer comprises a silicone elastomer, a diene elastomer, a butyl rubber, a natural rubber, a polyurethane rubber, an ethylene propylene diene monomer rubber, an isoprene rubber, a nitrile rubber, a styrene butadiene rubber, a blend, or combination thereof.

Embodiment 10. The plasma welding apparatus of any of Embodiments 1 to 9, wherein the operation chamber comprises an enclosure having at least one transparent surface through which the first profile and the second profile are visible.

Embodiment 11. The plasma welding apparatus of any of Embodiments 1 to 10, wherein the operation chamber comprises an enclosure having multiple transparent surfaces through which the first profile and the second profile are visible.

Embodiment 12. The plasma welding apparatus of any of Embodiments 10 to 11, wherein the transparent surface(s) at least partially blocks ultraviolet light from entering or exiting the enclosure.

Embodiment 13. The plasma welding apparatus of any of Embodiments 1 to 12, wherein the operation chamber forms a substantially sealed environment.

Embodiment 14. The plasma welding apparatus of any of Embodiments 1 to 13, wherein the operation chamber confines the plasma treatment within the substantially sealed environment.

Embodiment 15. The plasma welding apparatus of any of Embodiments 1 to 14, wherein the operation chamber comprises a ventilation system that provides a two-way air exchange with the operation chamber.

Embodiment 16. The plasma welding apparatus of Embodiment 15, wherein the ventilation system comprises a filter, a catalytic converter, a radiative element, or a combination thereof that treats the atmospheric air prior to the air entering or exiting the operation chamber.

Embodiment 17. The plasma welding apparatus of any of Embodiments 1 to 16, wherein the operation chamber is prevented from opening or removal during application of the plasma treatment.

Embodiment 18. The plasma welding apparatus of any of Embodiments 1 to 17, wherein the plasma generator comprises a gas supply and a power supply.

Embodiment 19. The plasma welding apparatus of Embodiment 18, wherein the gas supply comprises an atmospheric air supply, a compressor, a compressed gas cylinder, an in-house gas line, an in-house compressed gas line, a fan, a turbo, or a combination thereof.

Embodiment 20. The plasma welding apparatus of Embodiment 19, wherein the gas supply provides a flow of gas comprising an inert gas, an oxygen containing gas, a nitrogen containing gas, a fluorine containing gas, or a combination thereof.

Embodiment 21. The plasma welding apparatus of Embodiment 20, wherein the inert gas comprises argon, neon, helium, or any combination thereof.

Embodiment 22. The plasma welding apparatus of Embodiment 20, wherein the oxygen containing gas comprises atmospheric air, pure oxygen, alcohol, water vapor, or a combination thereof

Embodiment 23. The plasma welding apparatus of Embodiment 20, wherein the nitrogen containing gas comprises atmospheric air, pure nitrogen, ammonia, or a combination thereof.

Embodiment 24. The plasma welding apparatus of Embodiment 20, wherein the fluorine containing gas comprises sulfur hexafluoride (SF6), trifluoromethane (CHF3), tetrafluoromethane (CF4), octafluorocyclobutane (C4F8), or a combination thereof.

Embodiment 25. The plasma welding apparatus of Embodiment 18, wherein the power supply ionizes the flow of gas to generate the plasma treatment.

Embodiment 26. The plasma welding apparatus of Embodiment 25, wherein the power supply provides at least 110 VAC, at least 120 VAC, at least 220 VAC, or at least 240 VAC.

Embodiment 27. The plasma welding apparatus of Embodiment 25, wherein the power supply provides at least 6 VDC, at least 9 VDC, at least 12 VDC, at least 24 VDC, or at least 48 VDC.

Embodiment 28. The plasma welding apparatus of any of Embodiments 1 to 27, wherein the plasma generator comprises a supply line coupled to the at least one plasma head.

Embodiment 29. The plasma welding apparatus of any of Embodiments 1 to 28, further comprising: a plurality of plasma heads disposed within the operation chamber.

Embodiment 30. The plasma welding apparatus of Embodiment 29, wherein at least one of the plurality of plasma heads is disposed adjacently to the first profile within the operation chamber, and wherein at least one of the plurality of plasma heads is disposed adjacently to the second profile within the operation chamber.

Embodiment 31. The plasma welding apparatus of any of Embodiments 29 to 30, wherein at least one of the plurality of plasma heads is configured to sterilize the environment within the operation chamber.

Embodiment 32. The plasma welding apparatus of any of Embodiments 1 to 31, further comprising: a cutting device disposed in the operation chamber and configured to cut the first profile and the second profile.

Embodiment 33. The plasma welding apparatus of Embodiment 32, wherein the cutting device comprises at least one blade.

Embodiment 34. The plasma welding apparatus of Embodiment 33, wherein the at least one blade is heated, pre-sterilized, or a combination thereof.

Embodiment 35. The plasma welding apparatus of Embodiment 32, wherein the cutting device comprises a laser cutting system.

Embodiment 36. The plasma welding apparatus of any of Embodiments 1 to 34, further comprising: a mechanical motion module.

Embodiment 37. The plasma welding apparatus of Embodiment 36, wherein the mechanical motion module is configured to axially align the end contact surfaces of the first profile and the second profile.

Embodiment 38. The plasma welding apparatus of Embodiment 37, wherein the mechanical motion module linearly displaces at least one of the first profile and the second profile to axially align the end contact surfaces of the first profile and the second profile.

Embodiment 39. The plasma welding apparatus of Embodiment 37, wherein the mechanical motion module rotates at least one of the first profile and the second profile to axially align the end contact surfaces of the first profile and the second profile.

Embodiment 40. The plasma welding apparatus of any of Embodiments 36 to 39, wherein the mechanical motion module forces the end contact surfaces of the first profile and the second profile into contact to weld the first profile and the second profile together.

Embodiment 41. The plasma welding apparatus of any of Embodiments 36 to 40, wherein the first profile and the second profile are open during joining the first profile and the second profile.

Embodiment 42. The plasma welding apparatus of any of Embodiments 36 to 40, wherein the first profile and the second profile are closed prior to joining the first and the second profile.

Embodiment 43. The plasma welding apparatus of Embodiment 42, wherein the first profile and the second profile are closed at a distal end of each of the first profile and the second profile for plasma welding.

Embodiment 44. The plasma welding apparatus of Embodiment 42, wherein the first profile and the second profile are closed away from a distal end of each of the first profile and the second profile for plasma welding.

Embodiment 45. The plasma welding apparatus of any of Embodiments 1 to 44, further comprising: a control system.

Embodiment 46. The plasma welding apparatus of Embodiment 45, wherein the control system comprises a user interface and a plurality of sensors, indicators, gauges, or a combination thereof.

Embodiment 47. The plasma welding apparatus of Embodiment 46, wherein the user interface comprises a display.

Embodiment 48. The plasma welding apparatus of Embodiment 47, wherein the display is configured to display a temperature, a gas flow rate, a gas pressure, a gas detection level, a material of the first profile and the second profile, a plasma treatment progress level, a working cycle, a total number of working cycles, or a combination thereof.

Embodiment 49. The plasma welding apparatus of Embodiment 46, wherein the user interface comprises a material selection input for selecting properties a material of each of the first profile and the second profile.

Embodiment 50. The plasma welding apparatus of Embodiment 46, wherein the user interface comprises a warning system configured to alert a user when an out of conformance condition exists.

Embodiment 51. The plasma welding apparatus of Embodiment 50, wherein the user interface is configured to automatically stop the plasma treatment when an out of conformance condition exists.

Embodiment 52. The plasma welding apparatus of Embodiment 51, wherein the out of conformance condition comprises a low temperature, a high temperature, a low pressure, a high pressure, a low gas flow rate, a high gas flow rate, a detection of a plasma byproduct, an invalid selection of a material, a leak in the operation chamber, or a combination thereof.

Embodiment 53. The plasma welding apparatus of Embodiment 46, wherein the plurality of sensors comprises a temperature sensor, a gas flow rate sensor, a gas pressure sensor, a gas detection sensor, a plasma byproduct sensor, a tension sensor, or a combination thereof.

Embodiment 54. The plasma welding apparatus of any of Embodiments 45 to 53, wherein the control system comprises at least one vision system configured to verify axial alignment of the end contact surfaces of the first profile and the second profile, joining of the first profile to the second profile, or a combination thereof.

Embodiment 55. The plasma welding apparatus of any of Embodiments 45 to 54, wherein the control system is configured to conduct a burst test, a tension test, or a combination thereof between the first profile and the second profile after the first profile and the second profile are joined.

Embodiment 56. The plasma welding apparatus of any of Embodiments 45 to 55, wherein the control system is configured to store data related to operation of the plasma welding apparatus.

Embodiment 57. The plasma welding apparatus of any of Embodiments 1 to 56, wherein the plasma treatment activates material at the end contact surfaces of the first profile and the second profile.

Embodiment 58. The plasma welding apparatus of any of Embodiments 1 to 57, wherein the plasma treatment occurs between 10 degrees Celsius to 350 degrees Celsius.

Embodiment 59. The plasma welding apparatus of any of Embodiments 1 to 58, wherein the plasma treatment begins before cutting the first profile and the second profile.

Embodiment 60. The plasma welding apparatus of Embodiment 59, wherein the plasma treatment begins at least 1 second, at least 2 seconds, at least 3 seconds, at least 5 seconds, at least 10 seconds, at least 15 seconds at least 30 seconds, at least 45 seconds at least 60 seconds, at least 90 seconds, or at least 120 seconds before cutting the first profile and the second profile.

Embodiment 61. The plasma welding apparatus of any of Embodiments 1 to 60, wherein the plasma treatment continues after the first profile and the second profile are joined.

Embodiment 62. The plasma welding apparatus of Embodiment 61, wherein the plasma treatment continues for at least 1 second, at least 2 seconds, at least 3 seconds, at least 5 seconds, at least 10 seconds, at least 15 seconds at least 30 seconds, at least 45 seconds at least 60 seconds, at least 90 seconds, or at least 120 seconds after joining the first profile and the second profile.

Embodiment 63. The plasma welding apparatus of any of Embodiments 1 to 62, wherein a coincident weld formed between the first profile and the second profile comprises a tensile strength of at least 10%, at least 15%, at least 25%, at least 30%, at least 35%, at least 40%, at least 45%, or at least 50% of the tensile strength of an unmodified control bulk material of the first profile or the second profile.

Embodiment 64. The plasma welding apparatus of any of Embodiments 1 to 63, wherein a coincident weld formed between the first profile and the second profile comprises a burst pressure of at least 10%, at least 25%, at least 50%, at least 60%, at least 65%, at least 70%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, or at least 90% of the of the burst pressure of an unmodified control bulk material of the first profile or the second profile.

Embodiment 65. A method of operating a plasma welding apparatus, comprising: generating a plasma treatment with a plasma generator; applying the plasma treatment to an end contact surface of a first profile and an end contact surface of a second profile within an operation chamber; and manipulating at least one of the first profile and the second profile to force contact between the end contact surface of the first profile and the end contact surface of the second profile to join the first profile and the second profile.

Embodiment 66. The method of Embodiment 65, wherein the operation chamber forms a substantially sealed environment around the end contact surface of the first profile and the end contact surface of the second profile.

Embodiment 67. The method of Embodiment 65, further comprising: ionizing a flow of gas comprising an inert gas, an oxygen containing gas, a nitrogen containing gas, a fluorine containing gas, or a combination thereof to generate the plasma treatment.

Embodiment 68. The method of Embodiment 67, further comprising: sterilizing the environment within the operation chamber by applying the plasma treatment within the operation chamber.

Embodiment 69. The method of any of Embodiments 65 to 68, wherein the plasma treatment is applied within the operation chamber by at least one plasma head disposed within the operation chamber.

Embodiment 70. The method of any of Embodiments 65 to 69, further comprising: cutting the first profile and the second profile to expose the end contact surfaces of the first profile and the second profile.

Embodiment 71. The method of Embodiment 70, further comprising: applying the plasma treatment to the first profile and the second profile prior to cutting the first profile and the second profile.

Embodiment 72. The method of Embodiment 71, wherein the plasma treatment is applied to the first profile and the second profile at least 1 second, at least 2 seconds, at least 3 seconds, at least 5 seconds, at least 10 seconds, at least 15 seconds at least 30 seconds, at least 45 seconds at least 60 seconds, at least 90 seconds, or at least 120 seconds before cutting the first profile and the second profile.

Embodiment 73. The method of any of Embodiments 65 to 72, further comprising: applying the plasma treatment to the first profile and the second profile after joining the first profile and the second profile.

Embodiment 74. The method of Embodiment 73, wherein the plasma treatment is applied to the first profile and the second at least 1 second, at least 2 seconds, at least 3 seconds, at least 5 seconds, at least 10 seconds, at least 15 seconds at least 30 seconds, at least 45 seconds at least 60 seconds, at least 90 seconds, or at least 120 seconds after joining the first profile and the second profile.

Embodiment 75. The method of any of Embodiments 70 to 74, further comprising: axially align the end contact surfaces of the first profile and the second profile after cutting the first profile and the second profile.

Embodiment 76. The method of Embodiment 75, further comprising: verifying the axial alignment of the end contact surfaces of the first profile and the second profile via a camera system.

Embodiment 77. The method of any of Embodiments 75 to 76, wherein axially align the end contact surfaces of the first profile and the second comprises linearly displacing or rotating at least one of the first profile and the second profile.

Embodiment 78. The method of any of Embodiments 65 to 77, wherein the first profile and the second profile are open during joining the first profile to the second profile.

Embodiment 79. The method of any of Embodiments 65 to 77, further comprising: closing the first profile and the second profile prior to joining the first and the second profile.

Embodiment 80. The method of Embodiment 79, wherein the first profile and the second profile are closed at a distal end of each of the first profile and the second profile.

Embodiment 81. The method of Embodiment 79, wherein the first profile and the second profile are closed away from a distal end of each of the first profile and the second profile.

Embodiment 82. The method of any of Embodiments 65 to 81, further comprising: monitoring a temperature, a gas flow rate, a gas pressure, a gas detection level, a material of the first profile and the second profile, a plasma treatment progress level, a working cycle, a total number of working cycles, or a combination thereof.

Embodiment 83. The method of Embodiment 82, further comprising: displaying the temperature, the gas flow rate, the gas pressure, the gas detection level, the material of the first profile and the second profile, the plasma treatment progress level, the working cycle, the total number of working cycles, or a combination thereof on a user interface.

Embodiment 84. The method of any of Embodiments 82 to 83, further comprising: alerting a user when an out of conformance condition exists.

Embodiment 85. The method of Embodiment 84, further comprising: automatically stopping the plasma treatment when an out of conformance condition exists.

Embodiment 86. The method of Embodiment 85, wherein the out of conformance condition comprises a low temperature, a high temperature, a low pressure, a high pressure, a low gas flow rate, a high gas flow rate, a detection of a plasma byproduct, an invalid selection of a material, a leak in the operation chamber, or a combination thereof.

Embodiment 87. The method of any of Embodiments 65 to 86, further comprising: storing data related to operation of the plasma welding apparatus.

Embodiment 88. The method of any of Embodiments 65 to 87, further comprising: conducting a burst test, a tension test, or a combination thereof between the first profile and the second profile test after the first profile and the second profile are joined.

Embodiment 89. The method of Embodiment 83, further comprising: selecting a material of each of the first profile and the second profile via the user interface.

Embodiment 90. The method of any of Embodiments 65 to 89, wherein at least one of the first profile and the second profile are formed from a polymeric material.

Embodiment 91. The method of any of Embodiments 65 to 90, wherein each of the first profile and the second profile are formed from a polymeric material.

Embodiment 92. The method of any of Embodiments 90 to 91, wherein the first profile and the second profile are formed from the same polymeric material.

Embodiment 93. The method of any of Embodiments 90 to 91, wherein the first profile and the second profile are formed from different polymeric materials.

Embodiment 94. The method of any of Embodiments 65 to 93, wherein the first profile and the second profile are formed from a thermoplastic elastomer, a thermoset elastomer, or combination thereof.

Embodiment 95. The method of Embodiment 94, wherein the thermoplastic elastomer comprises a polystyrene, a polyester, a silicone copolymer, a silicone thermoplastic vulcanizate, a copolyester, a polyamide, a fluoropolymer, a polyolefin, a polyether-ester copolymer, a thermoplastic urethane, a polyether amide block copolymer, a polyamide copolymer, a styrene block copolymer, a polycarbonate, a thermoplastic vulcanizate, an ionomer, a polyoxymethylene (POM), an acrylonitrile butadiene styrene (ABS), an acetal, an acrylic, a polyvinyl chloride (PVC), a blend, or combination thereof.

Embodiment 96. The method of Embodiment 94, wherein the thermoset elastomer comprises a silicone elastomer, a diene elastomer, a butyl rubber, a natural rubber, a polyurethane rubber, an ethylene propylene diene monomer rubber, an isoprene rubber, a nitrile rubber, a styrene butadiene rubber, a blend, or combination thereof.

Embodiment 97. The method of any of Embodiments 65 to 96, wherein a coincident weld formed between the first profile and the second profile comprises a tensile strength of at least 10%, at least 15%, at least 25%, at least 30%, at least 35%, at least 40%, at least 45%, or at least 50% of the tensile strength of an unmodified control bulk material of the first profile or the second profile.

Embodiment 98. The method of any of Embodiments 65 to 97, wherein a coincident weld formed between the first profile and the second profile comprises a burst pressure of at least 10%, at least 25%, at least 50%, at least 60%, at least 65%, at least 70%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, or at least 90% of the of the burst pressure of an unmodified control bulk material of the first profile or the second profile.

Embodiment 99. The method of any of Embodiments 65 to 98, further comprising: applying a reinforcement at least partially about the coincident weld formed between the first profile and the second profile.

Embodiment 100. The method of Embodiment 99, wherein the reinforcement comprises an adhesive tape, a polymer tape, an overmolded polymer, a plasma-welded polymer, or a combination thereof.

Embodiment 101. The plasma welding apparatus of any of Embodiments 1 to 64 or the method of any of Embodiments 65 to 100, wherein applying the plasma treatment to the end contact surfaces of the first profile and the second profile comprises exposing or subjecting the end contact surfaces of the first profile and the second profile to the plasma treatment or directing the plasma treatment to contact or substantially envelope the end contact surfaces of the first profile and the second profile.

Embodiment 102. The plasma welding apparatus of any of Embodiments 1 to 64 or the method of any of Embodiments 65 to 101, wherein generating the plasma treatment is accomplished by generating ionized particles through a corona treatment, a C-treatment, a plasma treatment, a flame treatment, or any combination thereof.

Embodiment 103. The plasma welding apparatus of any of Embodiments 1 to 64 or the method of any of Embodiments 65 to 102, further comprising: a marking system configured to mark the first profile, the second profile, a joint formed between the first profile and the second profile, or a combination thereof via laser marking, ink marking, or any combination thereof that allows identification, verification, troubleshooting or any combination thereof of one or more characteristics of the joint between the first profile and the second profile.

Embodiment 104. The plasma welding apparatus of any of Embodiments 1 to 64 or the method of any of Embodiments 65 to 102, wherein applying the plasma treatment provides an interior environment of the operation chamber with a sterile environment, wherein the sterile environment is defined by reduction in an amount of living microorganisms within the interior environment of the operation chamber by a level of at least 106 after an exposure to the plasma treatment for at least 10 seconds, at least 15 seconds, at least 20 seconds, at least 25 seconds, or at least 30 seconds.

Embodiment 105. The plasma welding apparatus of any of Embodiments 1 to 64 or the method of any of Embodiments 65 to 102, wherein the operation chamber, the mechanical motion module, or a combination thereof is configured for mobile use and remotely coupled to the plasma generator, the control system, or a combination thereof.

Embodiment 106. The plasma welding apparatus of any of Embodiments 1 to 64 or the method of any of Embodiments 65 to 102, wherein the plasma welding apparatus comprises a unitary apparatus that is configured for mobile utility.

Embodiment 107. The plasma welding apparatus of any of Embodiments 1 to 31 or 36 to 64 or 101 to 106, further comprising: a mechanical motion module.

Embodiment 108. The plasma welding apparatus of Embodiment 107, wherein the mechanical motion module comprises a first cutting device and a second cutting device.

Embodiment 109. The plasma welding apparatus of Embodiment 108, wherein first cutting device is configured to cut a first tubing component to form the first profile and a second cutting device configured to cut a second tubing component to form the second profile.

Embodiment 110. The plasma welding apparatus of Embodiment 109, wherein the first cutting device is controlled by a first actuation device configured to move the first cutting device in a transverse direction to cut the first tubing component to form the first profile, and wherein the second cutting device is controlled by a second actuation device configured to move the second cutting device in an opposing transverse direction to cut the second tubing component to form the second profile.

Embodiment 111. The plasma welding apparatus of Embodiment 110, wherein the first cutting device and the second cutting device are aligned in the transverse direction.

Embodiment 112. The plasma welding apparatus of Embodiment 110, wherein the first cutting device and the second cutting device are misaligned in the transverse direction.

Embodiment 113. The plasma welding apparatus of any of Embodiments 108 to 110, wherein the first cutting device and the second cutting device cut the first profile and the second profile, respectively, at an angle less than 90 degrees with respect to a length of the first profile and the second profile.

Embodiment 114. The plasma welding apparatus of any of Embodiments 108 to 110, wherein the first cutting device and second cutting device cut the first profile and the second profile, respectively, substantially orthogonally with respect to a length of the first profile and the second profile.

Embodiment 115. The plasma welding apparatus of any of Embodiments 108 to 114, wherein the first actuation device is operated to retract the first cutting device to expose the end contact surface of the first profile to the plasma treatment after cutting the first tubing component to form the first profile, and wherein the second actuation device is operated to retract the second cutting device to expose the end contact surface of the second profile to the plasma treatment after cutting the second tubing component to form the second profile.

Embodiment 116. The plasma welding apparatus of any of Embodiments 108 to 115, wherein the first cutting device and the second cutting device are heated, pre-sterilized, or a combination thereof.

Embodiment 117. The plasma welding apparatus of any of Embodiments 107 to 116, wherein the mechanical motion module comprises a stationary tubing holder and a movable tubing holder.

Embodiment 118. The plasma welding apparatus of Embodiment 117, wherein the stationary tubing holder is configured to remain stationary and secure a first tubing component to be cut to form the first profile and a second tubing component to be cut to form the second profile.

Embodiment 119. The plasma welding apparatus of Embodiment 118, wherein the stationary tubing holder comprises adjustable tubing clamps that allow for various sized tubing components to be inserted within the stationary tubing holder.

Embodiment 120. The plasma welding apparatus of any of Embodiments 117 to 119, wherein the movable tubing holder is configured to secure the first tubing component to be cut to form the first profile and the second tubing component to be cut to form the second profile.

Embodiment 121. The plasma welding apparatus of Embodiment 120, wherein the movable tubing holder is configured to move both axially and transversally to axially align end contact surfaces of the first profile and the second profile after cutting the first component to form the first profile and the second component to form the second profile.

Embodiment 122. The plasma welding apparatus of Embodiment 121, wherein the first profile is secured in the movable tubing holder, and wherein the second profile is secured in the stationary tubing holder.

Embodiment 123. The plasma welding apparatus of Embodiment 122, wherein the movable tubing holder is controlled by a third actuation device configured to transversally move the movable tubing holder with respect to the stationary tubing holder to axially align the first profile and the second profile.

Embodiment 124. The plasma welding apparatus of any of Embodiments 117 to 123, wherein the second cutting device is controlled by a fourth actuation device configured to axially move the second cutting device to provide clearance for axial movement of the movable tubing holder.

Embodiment 125. The plasma welding apparatus of Embodiment 124, wherein the movable cutting device is controlled by a fifth actuation device configured to axially move the movable tubing holder to force the end contact surfaces of the first profile and the second profile into contact to join the first profile and the second profile.

Embodiment 126. The plasma welding apparatus of any of Embodiments 117 to 123, wherein the second cutting device is controlled by a fifth actuation device configured to axially move the second cutting to provide clearance for axial movement of the movable tubing holder, and wherein the movable tubing holder is simultaneously controlled by the fifth actuation device to axially move the movable tubing holder to force the end contact surfaces of the first profile and the second profile into contact to join the first profile and the second profile.

Embodiment 127. The plasma welding apparatus of any of Embodiments 107 to 126, wherein each of the actuation devices comprises a sensor that is coupled to the control system of the plasma welding apparatus.

Embodiment 128. The plasma welding apparatus of Embodiment 127, wherein the sensors are configured to relay information to the control system, and wherein the information comprises directional information, positional information, or other operational information.

Embodiment 129. The plasma welding apparatus of any of Embodiments 107 to 128, wherein the mechanical motion module comprises a plurality of tracks along which the first cutting device, the second cutting device, and the movable tubing holder are translated by their respective actuation devices.

Embodiment 130. The plasma welding apparatus of any of Embodiments 117 to 129, wherein the stationary tubing holder and the movable tubing holder are spaced apart a predetermined distance prior to cutting the first component to form the first profile and the second component to form the second profile.

Embodiment 131. The plasma welding apparatus of Embodiment 130, wherein the predetermined distance is at least 1.0 mm, at least 2.0 mm, at least 3.0 mm, at least 4.0 mm, or at least 5.0 mm.

Embodiment 132. The plasma welding apparatus of any of Embodiments 130 to 131, wherein the predetermined distance is not greater than 15.0 mm, not greater than 14.0 mm, not greater than 13.0 mm, not greater than 12.0 mm, not greater than 11.0 mm, or not greater than 10.0 mm.

Embodiment 133. The plasma welding apparatus of any of Embodiments 107 to 129, wherein the mechanical motion module comprises one or more alignment rods.

Embodiment 134. The plasma welding apparatus of Embodiment 133, wherein the stationary tubing holder comprises one or more alignment rods, and wherein the movable tubing holder comprises one or more alignment rods.

Embodiment 135. The plasma welding apparatus of Embodiment 134, wherein the one or more alignment rods of the stationary tubing holder and the one or more alignment rods of the movable tubing holder are configured to abut, engage, or otherwise interact to align the stationary tubing holder and the movable tubing holder.

Embodiment 136. The plasma welding apparatus of Embodiment 134, wherein the one or more alignment rods of the stationary tubing holder and the one or more alignment rods of the movable tubing holder are configured to selectively extend and guide the first profile and the second profile into contact with one another such that the end contact surfaces of the first profile and the second profile make contact and the first profile and the second profile are axially aligned.

Embodiment 137. The plasma welding apparatus of Embodiment 136, wherein the one or more alignment rods of the stationary tubing holder and the one or more alignment rods of the movable tubing holder extend to guide the first profile and the second profile into contact with one another such that the end contact surfaces of the first profile and the second profile make contact and the first profile and the second profile are axially aligned during application of the plasma treatment.

This written description uses examples to disclose the embodiments, including the best mode, and also to enable those of ordinary skill in the art to make and use the invention. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Also, the use of “a” or “an” are employed to describe elements and components described herein. This is done merely for convenience and to give a general sense of the scope of the invention. This description should be read to include one or at least one and the singular also includes the plural unless it is obvious that it is meant otherwise.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

After reading the specification, skilled artisans will appreciate that certain features are, for clarity, described herein in the context of separate embodiments, may also be provided in combination in a single embodiment. Conversely, various features that are, for brevity, described in the context of a single embodiment, may also be provided separately or in any subcombination. Further, references to values stated in ranges include each and every value within that range.

What is claimed is:

1. A plasma welding apparatus, comprising:
an operation chamber; and
a plasma generator having at least one plasma head disposed within the operation chamber and in proximity to an end contact surface of a first profile within the operation chamber and an end contact surface of a second profile within the operation chamber, wherein the plasma head is configured to apply a plasma treatment to the end contact surfaces of the first profile and the second profile to join the first profile and the second profile.

2. The plasma welding apparatus of claim 1, wherein the first profile and the second profile are formed from a thermoplastic elastomer, a thermoset elastomer, or combination thereof.

3. The plasma welding apparatus of claim 2, wherein the thermoplastic elastomer comprises a polystyrene, a polyester, a silicone copolymer, a silicone thermoplastic vulcanizate, a copolyester, a polyamide, a fluoropolymer, a polyolefin, a polyether-ester copolymer, a thermoplastic urethane, a polyether amide block copolymer, a polyamide copolymer, a styrene block copolymer, a polycarbonate, a thermoplastic vulcanizate, an ionomer, a polyoxymethylene (POM), an acrylonitrile butadiene styrene (ABS), an acetal, an acrylic, a polyvinyl chloride (PVC), a blend, or combination thereof.

4. The plasma welding apparatus of claim 2, wherein the thermoset elastomer comprises a silicone elastomer, a diene elastomer, a butyl rubber, a natural rubber, a polyurethane rubber, an ethylene propylene diene monomer rubber, an isoprene rubber, a nitrile rubber, a styrene butadiene rubber, a blend, or combination thereof.

5. The plasma welding apparatus of claim 1, wherein the operation chamber forms a substantially sealed environment.

6. The plasma welding apparatus of claim 5, wherein the operation chamber confines the plasma treatment within the substantially sealed environment.

7. The plasma welding apparatus of claim 1, wherein the operation chamber is prevented from opening or removal during application of the plasma treatment.

8. The plasma welding apparatus of claim 1, wherein the plasma generator comprises a gas supply and a power supply.

9. The plasma welding apparatus of claim 8, wherein the gas supply comprises an atmospheric air supply, a compressor, a compressed gas cylinder, an in-house gas line, an in-house compressed gas line, a fan, a turbo, or a combination thereof, and wherein the gas supply provides a flow of gas comprising an inert gas, an oxygen containing gas, a nitrogen containing gas, a fluorine containing gas, or a combination thereof.

10. The plasma welding apparatus of claim 9, wherein the inert gas comprises argon, neon, helium, or any combination thereof.

11. The plasma welding apparatus of claim 9, wherein the oxygen containing gas comprises atmospheric air, pure oxygen, alcohol, water vapor, or a combination thereof

12. The plasma welding apparatus of claim 9, wherein the nitrogen containing gas comprises atmospheric air, pure nitrogen, ammonia, or a combination thereof.

13. The plasma welding apparatus of claim 9, wherein the fluorine containing gas comprises sulfur hexafluoride (SF6), trifluoromethane (CHF3), tetrafluoromethane (CF4), octafluorocyclobutane (C4F8), or a combination thereof.

14. The plasma welding apparatus of claim 8, wherein the power supply ionizes a flow of gas from the gas supply to generate the plasma treatment.

15. The plasma welding apparatus of claim 1, wherein the plasma treatment activates material at the end contact surfaces of the first profile and the second profile.

16. The plasma welding apparatus of claim 1, further comprising: a plurality of plasma heads disposed within the operation chamber.

17. The plasma welding apparatus of claim 16, wherein at least one of the plurality of plasma heads is configured to sterilize the environment within the operation chamber.

18. The plasma welding apparatus of claim 1, further comprising: a cutting device disposed in the operation chamber and configured to cut the first profile and the second profile.

19. The plasma welding apparatus of claim 1, further comprising: a mechanical motion module configured to axially align the end contact surfaces of the first profile and the second profile.

20. The plasma welding apparatus of claim 1, further comprising: a control system configured to automatically stop the plasma treatment when an out of conformance condition exists.
